(19) United States
(12) Patent Application Publication
(10) Pub. No.: US 2007/0298016 A1
(43) Pub. Date: Dec. 27, 2007

(54) Methods For The Generation Of Hepatocyte-Like Cells From Human Blastocyst-Derived Stem (Hbs)

(76) Inventors: Nico Heins, Vastra Frolunda (SE); Henrik Semb, Bjarred (SE); Karolina Akesson, Gothenburg (SE); Marie Rehnstrom, Kallered (SE)

Correspondence Address: BUCHANAN, INGERSOLL & ROONEY PC, POST OFFICE BOX 1404, ALEXANDRIA, VA, 22313-1404, US

(21) Appl. No.: 11/663,091

(22) PCT Filed: Sep. 29, 2005
(86) PCT No.: PCT/EP05/10582

§ 371 Date: Sep. 5, 2007

Related U.S. Application Data
(60) Provisional application No. 60/613,965, filed on Sep. 29, 2004.

(30) Foreign Application Priority Data
Sep. 29, 2004 (DK) PA 2004 01489

(51) Int. Cl. A61K 35/00; C12N 5/08; C12Q 1/02

(52) U.S. Cl. 424/93.7; 435/29; 435/377

(57) ABSTRACT

The present invention relates to methods for obtaining endodermal progenitor cells and further differentiating these to hepatocyte-like cells. Knowledge about the cell composition prior to the initiation of terminal differentiation is used to select one of two different protocols and one of two type of intermediate progenitors depending on the purpose for which the resulting hepatocyte-like cells are needed. Protocol A of the present invention relates to differentiation of extraembryonic-resembling endodermal progenitor cells to hepatocyte-like cells and may be selected when yield and purity of the obtained hepatocyte-like cells is important. Protocol B of the present invention relates to differentiation of mesendodermal-resembling progenitor cells to hepatocyte-like cells and may be selected when quality of the obtained hepatocyte-like cells is important.

### FIELD OF THE INVENTION

The present invention concerns rapid, simple and efficient methods for the generation of hepatocyte-like cells, and the use of the hepatocyte-like cells obtained in the preparation of medicaments and for toxicity testings and in drug discovery and drug development.

### BACKGROUND OF THE INVENTION

Many diseases and disorders result from disruption of cellular function or destruction of tissues of the body. Today, donated organs and tissues are often used to replace failing or destroyed tissue. Unfortunately, the number of people suffering from disorders suitable for treatment by these methods far outstrips the number of organs available for transplantation.

The availability of human blastocyst-derived stem cells (hBS) and the intense research on developing efficient methods for guiding these cells towards different cell fates, e.g. endodermal cells, holds growing promise for future applications in cell-based treatments of such diseases. By reducing the need for organs such cell-based treatments are of great importance to both the society and to the individuals suffering from the above-mentioned diseases. Liver diseases or disorders caused by disruption of cellular function or destruction of tissues of the body is a major health problem to people all over the world.

Besides, the pharmaceutical industry today has a pronounced will to battle the escalating cost and time of drug discovery and development, and there is a growing need to increase the efficiency in the drug discovery process and to reduce late-stage attrition. Unexpected human metabolism is one of the major causes of removal of a potential new drug from a pharmaceutical project. In addition, liver toxicity and alterations of liver function is one of the most frequent occurring reasons for toxicology among drug molecules. Finally, liver metabolism and the interplay between hepatocytes and other organs are important drug targets for metabolic and dyslipidemic diseases. Traditionally, primary human hepatocytes have been isolated from cadavers or cancer resections. However, the supply of these cells is very limited and phenotypes vary widely among the sourced donors. Another disadvantage with primary hepatocytes is that they cannot be sustained in culture without losing function and thus their availability is dependent on repetitive sourcing. Due to these problems, pharmaceutical companies have to rely heavily on primary animal cells and transformed human cell lines for pre-clinical metabolism and toxicity testing, but the clinical relevance of such models and tests is usually low. Animal models are expensive and can only be performed in low-throughput and therefore such testing has been forced to be reserved for compounds in late pre-clinical development.

The availability of an unlimited source of functional human hepatocyte-like cells will provide tremendous advantages for drug discovery and development as well as for toxicity testings. Therefore, the development of improved human hepatocytes to be used for different in vitro assays will improve the quality of targets, hits, and leads; reduce late-stage attrition; and shorten time and cost of drug development. hBS cells are a potential novel source for functional human hepatocytes. These cells could be used in various human in vitro hepatocyte assays and would be an invaluable tool for both academic and industrial applications.

Today, the methods for generation of hepatocyte-like cells from hBS cells, which may be further differentiated into mature hepatocytes, often include the formation of embryoid bodies and/or early selection based on addition of cytotoxic compounds (Rambhatla et al., 2003). These selection steps, especially formation of embryoid bodies, result in a major cell number loss and in turn low efficiency. The methods are complicated, most having very long generation times and involve several time consuming steps. Thus, there is a need for rapid and simple method for the formation of hepatocyte-like cells derived from undifferentiated hBS cells. Previous attempts to obtain hepatocyte-like cells result in a low yield in relation to the starting material (US 20030003573).

In US20030003573 is further disclosed a method, wherein the cells are differentiated in a 2D culture without formation of EBs. However, the disclosed method leads to that more than 80% of the cells are lost within the first 24 hours.

### DESCRIPTION OF THE INVENTION

The present inventors have developed an improved method for generation of hepatocyte-like cells from hBS cells, the method taking advantage of the fact that dependent on the types of cells present in your starting material different conditions should be applied. In other words, knowledge about the composition of cells before an expansion step or a differentiation step, respectively, can improve the yield by adjusting said steps according to the composition of cells. Optionally the cells can be selected to enrich for either extraembryonic endodermal-resembling progenitor cells or embryonic mesendodermal-resembling progenitor cells in order to further improve the method.

Accordingly, the present invention provides methods for differentiating BS cells to hepatocyte-like cells without formation of EBs. The cells are cultured in dimensional culture comprising a surface to which the cells adhere. Accordingly, the present invention provides improved methods for differentiating BS cells in less time than existing methods. Furthermore, hepatocyte-like cells are generated by the methods according to the present invention without killing the majority of the cells within the first 24 hours.

Furthermore, the present inventors have found that knowledge about the cell composition prior to the initiation of differentiation can be used for selection of either of two different differentiation protocols depending on for what purpose the resulting hepatocyte-like cells are needed. Protocol A of the present invention, wherein primarily extraembryonic-resembling endodermal progenitor cells (endodermal progenitor cells of type A) are differentiated to hepatocyte-like cells, may be selected when yield and purity of the obtained hepatocyte-like cells is important. However, it is envisaged that hepatocyte-like cells obtained by differentiation of mesendodermal-resembling progenitor cells (endodermal progenitor cells of type B) may exhibit hepatocyte-like features to a higher extent than hepatocyte-like cells obtained by differentiation of extraembryonic-resembling endodermal progenitor cells. Thus, hepatocyte-like cells obtained via protocol B may express higher levels of the molecular markers used for identification of hepatocyte-like cells, than hepatocyte-like cells obtained via protocol A. Furthermore, hepatocyte-like cells obtained via protocol B may express a larger number of the molecular markers used for identification of hepatocyte-like cells, than hepatocyte-like cells obtained via pathway A. Accordingly, the present invention provides alternative methods for differentiating BS cells to hepatocyte-like cells taking advantage of the knowledge about the cell composition prior to initiation of differentiation in order to obtain hepatocyte-like cells satisfying different criteria with respect to yield, purity and quality. In the present context the yield is measured as the percentage of the number of hepatocyte-like cells obtained by the method with respect to the number of undifferentiated BS cells subjected to the method, purity is measured as the percentage of hepatocyte-like cells in the cell population obtained by the method and quality is measured by the expression levels of liver-specific markers and/or the number of liver-specific markers which is expressed simultaneously, where high expression levels and more simultaneously expressed liver-specific markers are indicative of good quality of hepatocyte-like cells. Identity of markers and the expression levels of these are compared to healthy, adult, primary hepatocytes.

With respect to the roughness of the treatment the intermediate progenitors are subjected to upon differentiation, the present inventors have found that it is beneficial to let the cells expand considerably before initiation of differentiation. Said expansion is achieved by letting cells grow for a longer ti me from as calculated from the time point of the initial plating of the BS cells in step i) to the time point where the final differentiation is induced in steps A-4) or B-4), whichever relevant. Furthermore, said expansion may be further stimulated by the addition of growth-promoting agents, such as, e.g., FGF 2 in step i) (both protocol A and B) and such as, e.g., retinoic acid (RA), FGF 4 and/or BMP2 in protocol A, and such as, e.g., activin A, HGF and/or Nodal for protocol B. These growth-promoting agents stimulates the formation of the respective type of endodermal progenitor cells desired for each of these protocols, i.e. addition of retinoic acid (RA), FGF 4 and/or BMP2 stimulates the formation of endodermal progenitor cells of type A and addition of HGF and/or Nodal stimulates the formation of endodermal progenitor cells of type B.

The method for obtaining endodermal progenitor cells and further differentiating these to hepatocyte-like cells comprises the steps:
i) In vitro differentiating BS cells in a growth medium to obtain differentiated cells of which at least a fraction of them are endodermal progenitor cells,
ii) determining the fraction of the endodermal progenitor cells obtained in step i) being extraembryonic-resembling endodermal progenitor cells (endodermal progenitor cells of type A) and/or mesendodermal-resembling progenitor cells (endodermal progenitor cells of type B),
iii) optionally, determining the fraction of the cells obtained in step i) being undifferentiated BS cells,
iv) optionally, selecting either endodermal progenitor cells of type A or endodermal progenitor cells of type B from the cell populations obtained in step i),
v) subjecting the endodermal progenitor cells of known composition obtained in steps i) or iv) to either protocol A or B, described herein
   in order to obtain hepatocyte-like cells.

In a specific embodiment, the BS cells are hBS cells.

In a further specific embodiment, step iv) is included.

The present inventors found that by subjecting the cells to different protocols of differentiation depending on the cell composition in step i) or iv), if relevant, the overall yield determined as the percentage of the number of hepatocyte-like cells obtained in proportion to the number of cells subjected to the method is improved and is at least 10%, such as, e.g., at least 20%, at least 30%, at least 40%, at least 50%, at least 60%, at least 70%, at least 80% or at least 90%.

During embryo development the extraembryonic endoderm is formed much earlier than the definitive endoderm, (here referred to as the embryonic endoderm). The extraembryonic endoderm gives rise to the yolk sac tissue and does never contribute to the embryo except as a source of signals, such as transcription factors and other peptides. The yolk sac serves in the first days as a proto liver and contains cell types with characteristics similar to those of cells that can be found later in the liver. The embryonic endoderm, such as the intermediary step mesendoderm on the other hand generates the internal organs lung, gut, pancreas and liver. It is formed by complex cell interactions and signals during gastrulation from a mesendodermal cell population that can give rise to mesoderm and endoderm. Growth factors and key genes decide which germ layer is being formed (Wells and Melton, 2000; Kubo et al., 2004).

Initial experiments performed in our lab following previously described protocols (Carpenter et al) (US20030003573) failed and resulted in very low yields of hepatocyte-like cells. Neither was any expandable progenitor population identified. In order to further improve the physiological quality of the experimental outcome, efforts have been made to identify the factors leading to improved results in order to obtain hepatocyte-like cells. As will be explained in more details below, the present inventors have found that it is important to identify or have knowledge about the starting material in order to choose the right conditions for the cells to be subjected to. Importantly, two different protocols have been developed depending on the composition of the endodermal progenitor cells used as starting material with respect to end odermal progenitors cells of type A and B.

The methods of the present invention therefore relates to two protocols to generate hepatocyte-like cells:

via the extraembryonic-resembling (primitive-like) endoderm protocol (A)

via the mesendodermal-resembling (also denoted the embryonic (definitive) endoderm-like) protocol (B).

In the following is given a list of definitions and abbreviations as used in the present context.

Definitions

As used herein, the term “blastocyst-derived stem cells” is used to describe pluripotent stem cells derived from the fertilized oocyte, i.e. the blastocyst. Pluripotency tests have shown that blastocyst-derived stem cells can give rise to all cells in the organism, including the germ cells. By the term “Blastocyst-derived stem cells” is also intended to mean what have traditionally been termed “embryonic stem cells”. However, according to many national laws in Europe and other countries, a fertilized oocyte is not regarded as an embryo within the first 14 days after fertilization. Since the blastocyst-derived cells employed according to the invention, are derived from the blastocyst 4-5 days after fertilization they are referred to as “blastocyst-derived stem cells” and not “embryonic stem cells”, the latter term being misleading with respect to the origin of these cells.

As used herein, the term “heptatocyte-like cells is used to describe cells that have a hepatocyte-like phenotype as measured by morphology and/or by positive reaction for one or more of the following markers: albumin, LFABP, AFP, AAT, CK 18, CYP and/or ASGPR.

As used herein, the terms “extraembryonic endoderm”, “extraembryonic-resembling endoderm” and “primitive endoderm” used interchangeably is intended to mean the early formed endoderm giving rise to the yolk sac tissue that never contributes directly to the embryo.

As used herein, the terms “extraembryonic endodermal progenitor cells”, “extraembryonic-resembling endodermal progenitor cells”, “primitive-like endodermal progenitor cells” and “endodermal progenitor cells of type A”, used interchangeably, is used to describe cells sharing characteristics with the in vivo developing primitive endoderm, as measured by reactions for either one of HNF3beta (hepatocyte nuclear factor 3), Gata4, Cdx2 (caudal-related homeobox transcription factor), Sox 17 (gene product of Sry-box containing gene 17), and Pdx1 (pancreatic duodenal homeobox factor-1) together with Oct-4.

As used herein, the terms “embryonic endoderm”, “mesendodermal endoderm”, “definitive endoderm” and “definitive-resembling endoderm”, used interchangeably, are intended to mean the different steps of endodermal development in the early developmental biology finally giving rise to the internal organs lung, gut, pancreas and liver. The terms “embryonic endodermal progenitor cells”, “mesendodermal progenitor cells”, “mesendodermal-resembling progenitor cells”, and “endodermal progenitor cells of type B”, used interchangeably, are then intended to mean cells sharing characteristics with the in vivo developing mesendoderm, as measured by co-localization of_positive reactions for Brachyury and HNF3beta.

By the terms “feeder cells” or “feeders” are intended to mean cells of one type that are co-cultured with cells of another type to provide an environment in which the cells of the second type can grow. The feeder cells may optionally be from the same or from a different species than the cells they are supporting, such as, e.g. human or mouse feeder cells supporting hBS cells. Further the feeder cells may optionally be committed towards a specific germ layer. The feeder cells may typically be inactivated when being co-cultured with other cells by irradiation or treatment with an anti-mitotic agent such as mitomycin c, to prevent them from outgrowing the cells they are supporting.

By the terms “feeder cell free” or “feeder free” is intended to mean cultures or cell populations wherein less than 5% of the total cells in the culture are feeder cells, such as, e.g., less than 4%, less than 3%, less than 2%, less than 1%, less than 0.5%, less than 0.1% and less than 0.01%. It will be recognized that if a previous culture containing feeder cells is used as a source of hBS for the culture to which fresh feeders are not added, there will be some feeder cells that survive the passage. However, after the passage the feeder cells will not grow, and only a small proportion will be viable by the end of 6 days of culture.

By the term “two-dimensional” culture is intended to mean culture conditions whereby the BS cells are proliferating and/or differentiating attached to a surface without involving embryoid body formation. These type of cultures are also referred to as adherent monolayer cultures.

As used herein, the term “medium change” means changing a volume between 10 and 100% of the used culture medium to fresh medium. A preferred medium change is about 50% of the volume in order to avoid exposing the cell cultures to osmotic stress.

As used herein the terms “liver-specific markers” or “markers used for identification of hepatocyte-like cells is intended to mean molecular markers known to be specifically expressed in hepatocytes in the body. Examples of such markers are albumin, LFABP, AFP, AAT, CK 18, CYP and ASGPR.

Abbreviations

As used herein, the term “blastocyst-derived stem cell” is denoted BS cell, and the human form is termed “hBS cells”.

As used herein the term “BMP”, such as the subtype “BMP2”, is intended to mean member of the bone morphogenic protein family that have important instructive functions in the early development, as for gastrulation and organogenesis.

As used herein, the term “RA” means retinoic acid.

As used herein, the term “AAT” is intended to mean the liver marker alpha-anti-trypsin.

As used herein, the term “AFP” is intended to mean the liver marker alpha-feto-protein.

As used herein, the term “CK18” is intended to mean the liver marker cytokeratin 18.

As used herein, the term “LFABP” means liver fatty acid binding protein.

As used herein, the term “ASGPR” is intended to mean asialoglycoprotein receptor, a trans-membrane protein mainly expressed in hepatocytes.

As used herein, the term “FGF” means fibroblast growth factor, preferably of human and recombinant origin, and belonging to subtypes fibroblast growth factor 2, fibroblast growth factor 4 and so forth.

As used herein, the term “HGF” means hepatocyte growth factor, sometimes referred to as scattered factor, SF in the literature.

As used herein, the term “DMSO” means dimethylsulfoxide.

As used herein, the term EBs or “embryoid bodies” is a term that is well defined within the field of stem cell research.

As used herein, the term “EF cells” means “embryonic fibroblast feeder”. These cells could be derived from any mammal, such as mouse or human.

As used herein “CYP” is intended to mean Cytochrome P, and more specifically Cytochrome P 450, the major phase 1 metabolizing enzyme of the liver constituting of many different subunits.

As used herein the term “OATP” is intended to mean Organic Anion Transporting polypeptide, that mediate the sodium (Na+)-independent transport of organic anions, such as sulfobromophthalein (BSP) and conjugated (taurocholate) and unconjugated (cholate) bile acids (by similarity) in the liver.

As used herein the “UGT” is intended to mean Uridine diphosphoglucuronosyltransferase, which is a group of liver enzymes catalyzing glucuronidation activities.

As used herein the “HNF3beta”, and/or “HNF3b”, used interchangeably are intended to mean hepatocyte nuclear factor 3, a transcription factor regulating gene expression in endodermal derived tissue, e.g. the liver, pancreatic islets, and adipocytes. HNF3beta is sometimes also referred to as Foxa2, the name originating from the transcription factor being a member of Forkhead box transcription factors family.

As used herein the “Cdx2” is intended to mean caudal-related homeobox transcription factor, which is known to play an important role in the regulation of cell proliferation and differentiation of e.g. the intestinal epithelium.

As used herein “Sox 17” is intended to mean the early endodermal marker, Sry-box containing gene 17, belonging to the family of genes which encode transcription factors with high-mobility-group DNA binding domain with diverse roles in development.

As used herein the “Pdx1” is intended to mean pancreas duodenum homeobox-1 transcription factor, sometimes also referred to as insulin promotor factor-1, islet/duodenum homeobox-1 etc, known to be expressed in e.g. duodenum, and pancreas, and more specifically in endocrine pancreatic cells.

The starting material in step i) is BS cells such as pluripotent/undifferentiated hBS cells, especially hBS cells. These BS cells can be obtained from a BS cell line, especially an hBS cell line. Although the present invention concerns hBS cells it cannot be excluded that a method according to the invention also can be applicable to other mammalian BS cells. hBS cells suitable for use in methods according to the invention can be obtained by the method e.g. described in WO03055992, which is hereby incorporated by reference. The BS cells may be propagated in a feeder-free or feeder culture system as described below.

In any step of the method and also prior to step i) the cells can be cultured in a 2 dimensional culture comprising a surface to which the cells adhere, such as on feeder cells or in a feeder free culture. It is contemplated that the culture system can be changed at any step of the method.

In a specific embodiment of the invention, the hBS cells used as starting material have at least one of the following properties

a) exhibit proliferation capacity in an undifferentiated state for more than 12 months when grown on mitotically inactivated feeder cells or under feeder free growth conditions,

b) exhibit and maintain their karyotype with chromosomes of human feature,

c) maintain potential to develop into derivatives of all types of germ layers both in vitro and in vivo,

d) exhibit at least two of the following markers OCT-4, Nanog, alkaline phosphatase, the carbohydrate epitopes SSEA-3, SSEA-4, TRA 1-60, TRA 1-81, and the protein core of a keratin sulfate/chondroitin sulfate pericellular matrix proteinglycan recognized by the monoclonal antibody GCTM-2,

e) do not exhibit molecular marker SSEA-1 or other differentiation markers, and

f) retain their pluripotency and form teratomas in vivo when injected into immuno-compromised mice,

g) are capable of differentiating into derivatives of all three germ layers.

In one embodiment the hBS cells have all the properties mentioned above.

The method relies on the early differentiation process that creates the diversity of cell populations that can respond to the factors that induce the different types of endodermal differentiation. As appears from the following description of the protocols A and B described herein, there may be differences in the initial differentiation step dependant on whether hepatocyte-like cells are prepared with extraembryonic-resembling endodermal progenitor cells (endodermal progenitors type A) or mesendodermal-resembling progenitor cells (endodermal progenitors type B), respectively, as starting material.

Generation of Endodermal Progenitor Cells of type A and Further Differentiation to Hepatocyte-Like Cells (Protocol A)

As mentioned above, the endodermal progenitor cells obtained in step i) or iv), if relevant, may be subjected to protocol A as described herein. In one embodiment, protocol A is employed when the fraction contains endodermal progenitor cells of type A, in particular when the fraction of endodermal progenitor cells of type A obtained in steps i) or iv) is larger than the fraction of endodermal progenitor cells of type B obtained in steps i) or iv). Protocol A is typically chosen, when the yield of hepatocyte-like cells compared to the undifferentiated BS cells initially subjected to the method, and/or the purity of the of the obtained hepatocyte-like cells are the most important determinants.

Protocol A according to the invention comprises the following steps:
A-1) subjecting the endodermal progenitor cells of known composition obtained in steps i) or iv), if relevant, to a growth medium and optionally, changing the growth medium after suitable period(s) of time,
A-2) expansion of the endodermal progenitor cells of known composition obtained in steps i), iv) or A-1) by addition of one or more growth-promoting agents,
A-3) optionally, passaging the cells obtained in steps i), iv) or A-2) one or more times leading to further expansion of said cells,
A-4) induction of differentiation of the progenitor cells obtained in steps i), iv), A-2) or A-3) by addition of one or more differentiating agents
   to obtain hepatocyte-like cells

In one embodiment of the present invention the cells in steps i), v), and A-1)-A-4) are cultured in a 2 dimensional culture comprising a surface to which the cells adhere.

Initial Differentiation

In order to obtain endodermal progenitors cells of type A, the initial treatment of the BS cells involving initial differentiation in step i) may be performed by addition of FGF 2 to the growth medium in step i). FGF 2 may be added to a concentration from about 0.1 ng/ml to about 200 ng/ml, such as, e.g., from about 0.5 ng/ml to about 100 ng/ml, from about 1 ng/ml to about 50 ng/ml, from about 1 ng/ml to about 25 ng/ml, from about 2 ng/ml to about 20 ng/ml, from about 2 ng/ml to about 10 ng/ml, from about 3 ng/ml to about 5 ng/ml.

The BS cells employed as starting material in step i) may be BS cells which have been cultured in the presence or absence of feeder cells prior to the initial differentiation (subjection to step i).

When feeder cells are present, the BS cells, especially the hBS cells, may be kept on feeder cells, such as mouse EF cells or human feeder cells without medium change from about 2 to 14 days, such as from 3 to 12 days, from 4 to 11 days, from 5 to 10 days, from 6 to 9 days, from 7 to 8 days. The culture medium can be changed after about 2 to 14 days, such as after 3 to 12 days, from 4 to 11 days, from 5 to 10 days, from 6 to 9 days, from 7 to 8 days and switched to a supplemented medium promoting extraembryonic endoderm development.

Alternatively, the hBS cells may be cultured in feeder-free medium e.g. on any suitable support matrix, such as Matrigel™ without passage or medium change from about 2 to 28 days, such as from 4 to 25 days, from 6 to 20 days, from 8 to 18 days, from 10 to 16 days, from 12 to 14 days. The culture medium can then be changed to a supplemented medium promoting extraembryonic endoderm development.

To confirm that the progenitors obtained are endodermal progenitor cells of type A, a fraction of those can be characterized by positive reactions for markers such as Oct-4, Pdx-1, HNF3b, and negative reactions for markers specific for undifferentiated hBS cells such as e.g. SSEA-4, Tra1-81, Tra1-60.

The fraction of the cells obtained in step i) and/or step iv) that are endodermal progenitor cells of type A is at least 10% such as, e.g., at least 15%, at least 20%, at least 25%, at least 30%, at least 40%, at least 50%, at least 60%, at least 70%, at least 80%, at least 90% as evidenced in a sample of these cells.

Furthermore, the fraction of the cells obtained in step i) and/or step iv) that are undifferentiated BS cells is less than 85% such as, e.g., less than 70%, such as, e.g., less than 60%, less than 50%, or less than 40% as evidenced in a sample of these cells.

In one embodiment of the present invention, endodermal progenitor cells of type A obtained in step i) are selected by inclusion of step iv).

Endodermal progenitors, such as endodermal progenitors of type A, may be selected by generating reporter gene BS cell lines obtained by either transgenic constructs or homologous recombination, in which the expression of a reporter gene, such as eGFP (enhanced green fluorescent protein) and/or DsRed (Discosoma sp. red fluorescent protein) under control of extraembryonic relevant promoters, such as PDX-1, Oct-4, HNF-3b or any suitable endodermal promoter or combinations thereof. The selection can thereafter be performed using e.g. neomycin selection, whereby the cells taking up the introduced resistance gene survive in a culture system with the antibiotic neomycin present or by dissociating the cells and sort them based on their expression of the reporter gene by e.g flow cytometry (FAC sorting). In this way endodermal progenitor cells of type A can be selected by

a) using neomycin selection in culture, and/or

b) using flow cytometry.

A transgenic hBS cell line can be generated by genetic engineering by e.g. transfection or any suitable method, such as lipofectamine, a lentiviral vector, or electroporation to introduce the DNA comprising the marker gene of interest and thereby obtain transient and/or stable expression of proteins of interest under the control of tissue-specific promoters.

Expansion and Passaging of the Extraembryonic Endodermal Progenitor Cells

The expansion of endodermal progenitor cells of type A may be on feeder cells or in a feeder-free culture system.

When the endodermal progenitor cells of type A are cultured in the presence of feeder cells the cells may be cultured in the promoting medium for a time period of from about 2 to about 14 days, such as from about 3 to about 13 days, from about 4 to about 12 days, from about 5 to about 11 days, from about 6 to about 10 days, from about 7 to about 9 days and, optionally, the medium may be changed once a week. The endodermal progenitor cells obtained after step i), step A-1) and/or A-2) can be dissected and re-plated on fresh feeder after from about 2 to about 14 days in culture, such as after from about 3 to about 13 days, after from about 4 to about 12 days, after from about 5 to about 11 days, after from about 6 to 10 days, after from about 7 to about 9 days, such as after about 8 days in culture. The dissection may be performed using any convenient instrument, such as a pipette or glass capillary. The subsequent passage (step A-3) may be performed using a chelator or enzymatic treatment after from about 2 to about 14 days, such as after from about 3 to about 13 days, after from about 4 to about 12 days, after from about 5 to about 11 days, after from about 6 to about 10 days, after from about 7 to about 9 days, such as after about 8 days in culture and the cells further transferred to fresh feeder, any culture supporting matrix or plastic. Subsequent passages can be performed by enzymatic treatment after from about 2 to about 14 days, such as after from about 3 to about 13 days, after from about 4 to about 12 days, after from about 5 to about 1 1 days, after from about 6 to about 10 days, after from about 7 to about 9 days, such as after about 8 days in culture.

In one embodiment of the invention the endodermal progenitor cells of type A obtained in step ii) were dissected and re-plated on fresh mouse EF cells using a glass capillary as cutting and transfer tool. The following passage was performed using trypsinization and the cells further transferred to tissue culture treated plastic dishes under feeder-free conditions. All subsequent passages were performed using trypsin and the culture maintained on plastic for more than 12 passages.

When the endodermal progenitor cells of type A are cultured in a feeder-free culture system, the cells may be cultured in the promoting medium (the medium used for expansion) from about 2 to about 28 days, such as from about 4 to about 26 days, from about 6 to about 24 days, from about 8 to about 20 days, from about 10 to about 18 days, from about 11 to about 17 days, from about 12 to about 16 days, from about 13 to about 15 days, such as for about 14 days and the medium may be changed between 1 and 10 times during the period, such as between 2 and 9 times, between 3 and 8 times, between 4 and 7 times, between 5 and 6 times. The progenitor cells obtained after step i), step A-1) and/or A-2) can be transferred to a fresh culture system after 2 to 28 days in culture, such as after 4 to 24 days, after 6 to 20 days, after 8 to 16 days, after 9 to 14 days, after 11 to 12 days, such as after 8 days in culture. The transfer may be performed using any convenient instrument, such as a pipette or glass capillary or by enzymatic treatment or a chelator. Subsequent passages (step A-3) can be performed by enzymatic treatment after from about 2 to about 14 days, such as after from about 3 to about 13 days, after from about 4 to about 12 days, after from abut 5 to about 11 days, after from about 6 to about 10 days, after from about 7 to about 9 days, such as after about 8 days in culture.

Different compounds can be used to promote proliferation of extraembryonic cells (endodermal progenitor cells of type A). Examples of such compounds are for example retinoic acid, FGF4 and/or BMP2, which compounds selectively promote the proliferation of endodermal progenitor cells of type A. In one embodiment of the present invention, the one or more growth-promoting agents added in step A-2) is therefore selected from the group consisting of RA, FGF4, and BMP2. RA, FGF4, and/or BMP2 can be added to a concentration from about 0.1 ng/ml to about 1000 ng/ml, such as, e.g., from about 0.5 ng/ml to about 800 ng/ml, from about 1 ng/ml to about 600 ng/ml, from about 1.5 ng/ml to about 400 ng/ml, from about 2 ng/ml to about 200 ng/ml, from about 2.5 ng/ml to about 100 ng/ml, from about 3 ng/ml to about 50 ng/ml, from about 3 ng/ml to about 20 ng/ml, from about 3.5 ng/ml to about 20 ng/ml or from about 4 to about 8 ng/ml.

In one embodiment of the present invention the fraction of the cells obtained in step A-2) that are endodermal progenitor cells of type A is at least 20%, such as, e.g., at least 30%, at least 40%, at least 50%, at least 60%, at least 70%, at least 80%, at least 85% or at least 90% as evidenced in a sample of these cells. Endodermal progenitor cells of type A can be identified by positive reaction for any early expressed endodermal marker, such as a marker selected from the group consisting of HNF3beta, Gata4, Cdx2, Sox 17 and Pdx1.

To confirm that the progenitors obtained are endodermal progenitors of type A, a fraction of those can be characterized by positive reaction for the marker Oct-4 or positive reactions for markers such as Oct-4 together with any of the markers Gata-4, Cdx2, Sox17, Pdx-1 and HNF3b, and negative reactions for markers specific for undifferentiated hBS cells such as e.g. SSEA-4, Tra1-81, Tra1-60 and/or Nanog.

In one embodiment of the present invention the endodermal progenitor cells of type A obtained in step i), iv) or A-2) are identified by positive reaction of at least one of the following markers, such as, e.g., at least two of the following markers, at least three of the following markers, at least four of the following markers, at least five of the following markers HNF3beta, Gata4, Cdx2, Sox17, and Pdx1 or by positive reaction for HNF3beta, Gata4, Cdx2 and Pdx1.

As mentioned above, in specific embodiments, step A-3) is included and the endodermal progenitor cells of type A obtained in step i) or iv), if relevant, and/or expanded in step A-2) can be further propagated on either feeder layers or in a feeder free culture system, and passaged by either mechanical dissection, enzymatic treatment or by using a mild chelator treatment such as EDTA.

The cell population obtained may further be characterized by the co-localization of Oct-4 and Pdx-1.

In general, the population of endodermal progenitor cells of type A is increased with a factor of at least 2 such as, e.g., a factor or 10 or more, a factor of 50 or more, a factor of 100 or more, a factor of 250 or more, a factor of 500 or more, a factor of 750 or more or a factor of 1000 or more after step A-2) or A-3).

Differentiation of the endodermal progenitor cells of type A to hepatocyte-like cells To carry out step A-4), the medium is changed to a differentiation medium containing one or more differentiating agents in which the cells can be cultured for between 2 to 14 days, such as between 3 and 12 day, between 4 and 11 day, between 5 and 10 day, between 6 and 19 days, between 7 and 8 days. However, if the cells have been cultured in a feeder-free culture system, the cultivation in the presence of one or more differentiating agents may be up to 60 days such as from about 5 to about 50 days, from about 10 to about 40 days or about 30 days. Suitable examples of one or more differentiating agents are toxic agents, especially toxic agents that can be degraded by the liver. The differentiating agent can be an alcohol, such as, e.g. ethanol, or it may be DMSO, dexamethasone, Phenobarbital, Urea or combinations thereof.

In a preferred embodiment the differentiating agent is DMSO added to a concentration from about 0.5% to about 10%, such as, e.g., from about 0.5% to about 9%, from about 0.6% to about 8%, from about 0.6% to about 7%, from about 0.7% to about 6%, from about 0.7% to about 5%, from about 0.7% to about 4%, from about 0.8% to about 3%, from about 0.8% to about 2%, from about 0.8% to about 1.8%, from about 0.8% to about 1.6%, from about 0.9% to about 1.4%, from about 0.9% to about 1.2%, from about 0.9% to about 1.1%.

The cell population obtained may display liver markers such as alpha-fetoprotein (AFP), alpha-antitrypsin (AAT), liver fatty acid binding protein (LFABP), cytokeratin 18 (CK18), albumin and asialoglycoproteinreceptor (ASGPR) in combination with low or no expression of endodermal specific markers, such as, e.g., HNF3b.

Further characterization such as gene profiling and functional tests based on e.g. liver specific enzymatic activity can also be applied as well as transplantation of the endodermal progenitor cells and/or the hepatocyte-like cells to hepatectomic mice.

The major phase 1 biotransformation or metabolising system is the cytochrome P 450 (CYP). CYP expression in the different cell types obtained in the present invention can be analyzed on protein level by immunohistochemistry and/or Western Blot. An additional method is to genetically analyze the cells obtained in the present invention using real time PCR. Specific CYP subtypes of interest can be CYP 3A4 and CYP 1A2 (abundant in human liver), and CYP 3A7 (expressed in fetal liver tissue). CYP induction can then be tested by adding different known inducers, such as e.g. dexamethasone, rifampicin, and/or omeprazole.

The progenitor cells and hepatocyte-like cells obtained rnay further be analyzed for Glutathione transferases (GSTs). GSTs are phase II biotransformation enzymes that catalyze the conjugation of electrophilic xenobiotics with glutathione (GSH). Since GSTs have a wide range of substrates and GSH is highly abundant, GSTs are important players in detoxification of xenobiotics. Analysis of Phase II enzyme induction can be performed by any suitable method, such as immunofluorescence, confocal microscopy and/or possibly western blot analysis.

Multidrug resistance protein or P-glycoprotein is a transport protein that exports anionic conjugates and other substrates from the cell. Characterization of this third phase of the detoxification process could contribute to a more complete picture c)f metabolism of the different cell types obtained in the present invention.

The hepatocyte-like cells and/or intermediary progenitor cells, such as, e.g., endodermal progenitor cells of type A or B, obtained in the present invention may as well be characterized by analyzing their expression profile of metabolically significant genes. In the following is described how this can be done. RNA can be isolated from undifferentiated and differentiated hBS cells, such as endodermal progenitor cells and hepatocyte-like cells and, as a positive control, human liver RNA. RNA extracted from a hepatic cell line, such as HepG2 may as well be used as a control. An expression profile can then be obtained comparing many different genes by using any suitable genetic tool for analysis, such as micro arrays, followed by bioinformatics analysis using a suitable software. The different cell types of the present invention may then show more or less expression of key genes, i.e. any genes which are specifically expressed in human adult liver, such as genes encoding for liver specific enzymes and transporter proteins. In addition the expression of the liver specific genes albumin and glucose-6-phosphatase can be analyzed for and possibly compared to the expression levels of the control samples.

Normally, the hepatocyte-like cells are identified by positive reaction for a marker selected of the group consisting of albumin, AFP, AAT, CK 18, LFABP, CYP and ASGPR. The positive reaction should be established for at least one of the following markers, such as, e.g., at least two of the following markers, at least three of the following markers, at least four, at least five of the following markers, at least six of the following markers: albumin, AFP, AAT, CK 18, LFABP, CYP and ASG PR. Especially, the hepatocyte-like cells may be identified by positive reaction for albumin, AFP, AAT, CK 18 and LFABP.

By using the method involving protocol A according to the invention, the fraction of the cells obtained in step A-4) that are hepatocyte-like cells is at least 5%, such as, e.g., at least 10%, at least 20%, at least 30%, at least 40%, at least 50%, at least 60%, at least 70%, at least 80%, at least 90% as evidenced in a sample of these cells.

The fraction of the cells obtained in step A-4) that are undifferentiated BS cells may be less than 2%, such as, e.g., less than 1%, less than 0.5% as evidenced in a sample of these cells.

When applying protocol A, the cells grow in two-dimensional cultures.

In a specific embodiment of the invention, the cells obtained in step A-1) are further propagated by inclusion of step A-2). The obtained cells may be passaged as in step A-3) at about 60-90% confluence, such as, e.g., at about 65-85% confluence, at about 70-80% confluence. If the progenitors are cultured for too long before passage, they will become too confluent, and there is an intrinsic change due to contact inhibition and/or differentiation. Step A-3) may be repeated from about 1 to about 30 times, such as, e.g., from about 5 to about 25 times, from about 10 to about 20 times.

In order to determine whether the cells obtained from any of the steps A1) to A-3) are committed to an endodermal cell fate, the cells have to fulfil several criteria based on immuncytochemistry and morphology. Furthermore they should express one or more endodermal specific markers like HNF3beta, gata4, Cdx2, Sox17 and Pdx1 or one or more liver cell marker like Albumin, AFP, AAT, CK 18, LFABP and/or ASGPR. Another test is the determination whether the cells still express markers for undifferentiated hBS cells, such as, e.g. the markers Nanog, SSEA-3, SSEA-4, GCTM-2, Tra-1-60, Tra-1-81, and/or Oct-4.

Spontaneous differentiation generates the variety of cells that can respond to the growth factors or low molecular compounds that we have used. The extraembryonic-resembling endoderm is the first endoderm that is present already after a few days, such as, e.g. three days in the cultures and can be identified by an Oct4-Pdx1-double-positive and SSEA-4 negative cell population.

In one embodiment of the invention, the cells obtained from A-1)-A-3) have at least one of the following properties:
I) a majority of the cell population expressing at least one of the following markers HNF3beta, Cdx2, Gata-4, Sox17 and/or Pdx1 together with Oct4
II) a majority of the cell population expressing at least one of the following markers HNF3beta, Gata4, Pdx1, Sox17
III) a majority of the cell population being capable of further differentiation with expression of at least one of the following markers Albumin, HNF3beta, LFABP, Ck18, AFP, AAT, CYP and ASGPR.
IV) a majority of the cell population being unable to express one or more of the following markers for undifferentiated hBS cells, SSEA-3, SSEA-4, GCTM-2, Tra1-60 Tra1-81 and Nanog.

In a specific embodiment of the invention, the cells obtained have all properties from I) to IV) above.

In the present context, the term “majority of cells” is intended to denote at least about 60% of the cells such as, e.g., at least about 75%, at least about 90% or at least about 95% of the cells.

It can also be assessed whether the cells express markers for cell types of other germ layers than endodermal, such as ectoderm and mesoderm. For this purpose nestin, GFAP, beta-III-tubulin (markers for ectoderm) as well as ASMA (alpha smooth muscle actin), Brachyury and Desmin (markers for mesoderm) can be used.

In one embodiment of the invention, the majority of the cells obtained from step A-1) and/or step A-2) and/or step A-3), do not express markers for the ectoderm, such as, e.g., GFAP or/and nestin.

The cells obtained from step A-2)-A-4) may be further differentiated into at least one of the liver cell lineages, e.g., oval cells or hepatocytes.

In one embodiment of the invention, the differentiated cells obtained in A-4) may express at least one of the following liver cell type markers, including at least one of the markers HNF3beta, albumin, AFP, AAT, CK18, LFABP, CYP and ASGPR.

The expression profiles and levels of specific genes or markers important for hepatocytes can be measured by e.g. RNA extraction and subsequent quantitative PCR, whereby the amount of any specific hepatic marker can be compared to control samples of e.g. traditionally used hepatic cell lines, such as, e.g., such as HepG2, and adult human hepatocytes. Hepatocyte-like cells obtained from endodermal progenitors of type A may show a profile in terms of which genes that are expressed and the expression levels of those genes similar to healthy, adult human hepatocytes.

Specifically they may share the same profile of markers expressed, such as e.g. at least 20%, at least 30%, at least 40%, at least 50%, at least 60%, at least 70% of the markers expressed in healthy, adult (human) hepatocytes.

The expression levels of the individual markers may further constitute at least 2%, such as, e.g., at least 5%, at least 10%, at least 20%, at least 30%, at least 40%, at least 50%, at least 60%, at least 70% of the levels expressed in healthy adult hepatocytes cultured under the same conditions.

The undifferentiated BS cells can be identified by positive reaction for a marker selected from the group consisting of Nanog, SSEA-3, SSEA-4, GCTM-2, Tra1-60, Tra1-81 and Oct-4 (this also applies for other steps in the method according to the method, where it is of relevance to identify any undifferentiated BS cells).

In a specific embodiment undifferentiated BS cells are identified by positive reaction for at least one of said markers, such as, e.g., at least two of said markers, at least three of said markers, at least four of said markers, at least five of said markers of the undifferentiated BS cells are identified by positive reaction for Nanog, SSEA-3, SSEA-4, GCTM-2, Tra1-60, Tra1-81 and Oct-4. In a specific embodiment the undifferentiated BS cells are identified by positive reaction for SSEA-3, SSEA-4, GCTM-2, Tra1-60, Tra1-81, Nanog and Oct-4.

Generation of Endodermal Progenitor Cells of Type B and Further Differentiation to Hepatocyte-Like Cells (Protocol B)

As mentioned above, the endodermal progenitor cells obtained in step i) or iv), if relevant, may be subjected to protocol B as described herein. In one embodiment, protocol B is employed when the fraction of cells obtained in step i) or iv), if relevant, that is mesendodermal progenitor cells is at least 0.5%, such as, e.g., at least 5%, at least 10%, at least 15%, at least 20%, at least 25% or at least 30%, as evidenced in a sample of these cells. Protocol B is typically chosen, when the quality of the hepatocyte-like cells is the most important criterion for selecting the differentiation protocol.

Protocol B comprises the following steps:
B-1) subjecting the endodermal progenitor cells of known composition obtained in step i) or iv), if relevant, to a growth medium and optionally, changing the growth medium after suitable period(s) of time,
B-2) expanding the endodermal progenitor cells of known composition obtained in step i), iv) or B-1) by addition of one or more growth-promoting agents,
B-3) optionally, passaging the cells obtained in step i), iv) or B-2) one or more times leading to further expansion of said cells,
B-4) inducing differentiation of the progenitor cells obtained in step i), iv), B-2) or B-3) by adding one or more differentiating agents
   to obtain hepatocyte-like cells.

In one embodiment of the present invention protocol B, the cells in step i), iv), and B-1) to B-4) are cultured in a 2 dimensional culture comprising a surface to which the cells adhere.

Initial Differentiation

The initial differentiation step i) is essentially identical to that described in protocol A.

The BS cells such as the hBS cells are cultured in the presence of feeder cells such as mouse EF cells or human fibroblasts without passage or medium change for between 3 and 20 days, such as between 4 and 18 days, such as between 5 and 15 days, such as between 6 and 12 days, such as between 7 and 10 days.

Alternatively, the BS cells may be cultured in a feeder-free culture system such as on a culture support matrix without passage or medium change for between 2 and 28 days, such as between 4 and 30 days, such as between 5 and 20 days, such as between 6 and 15 days, such as between 7 and 10 days.

The mesendodermal-resembling progenitor population (cell type B) may thereafter be identified by a e.g. suitable set of antibody stainings such as for the combination of Brachyury and HNF3b, which can be regarded as a criterion for mesendodermal progenitor cells (Kubo et al., 2004).

The initial differentiation process that creates the diversity of cell populations that can respond to the factors that induce the distinct differentiation. Growth-promoting agents, such as, e.g., Activin A, HGF, and Nodal can be used to selectively promote proliferation of endodermal progenitor cells of type B.

The fraction of the cells obtained in step i) and/or step iii) that is undifferentiated BS cells is less than 85% such as, e.g., less than 70%, less than 60%, less than 50%, or less than 40% as evidenced in a sample of these cells. Moreover, the fraction of the cells obtained in step i) and/or iii) that are ectodermal progenitor cells is less than 30% such as, e.g., less than 20%, less than 10% or less than 5% as evidenced in a sample of these cells. Ectodermal progenitor cells may be identified by positive reaction for one or more molecular markers specific for ectoderm, such as, e.g., GFAP (glial fibrillary acidic protein) and nestin.

The presence of mesendodermal progenitors in step i) and/ ii) above can be further confirmed by investigating the potential of the endodermal progenitors obtained in step i) and step iv) to differentiate into cell types of mesendodermal origin in vivo and/or in vitro.

The population of endodermal progenitor cells of type B obtained in step i) may be selected by inclusion of step iv).

Endodermal progenitors, such as progenitors of subtype B, may be selected by generating reporter gene BS cell lines obtained by either transgenic constructs or homologous recombination, in which the expression of a reporter gene, such as eGFP (green fluorescent protein) and/or dsRed (Discosoma sp. red fluorescent protein) is under control of mesendodermal relevant promoters, such as a combination of HNF3beta and Brachyury or any other suitable combinations. The selection can thereafter be performed using e.g. neomycin selection, whereby the cells taking up the introduced resistance gene survive in a culture system with the antibiotic neomycin present or by dissociating the cells and sort them based on their expression of the marker gene by e.g flow cytometry (FAC sorting). In this way endodermal progenitor cells of type B can be selected by

a) using neomycin selection in culture, and/or

b) using flow cytometry.

A transgenic hBS cell line can be generated by genetic engineering by e.g. transfection or any suitable method, such as lipofectamine, a lentiviral vector, or electroporation to introduce the DNA comprising the marker gene of interest and thereby obtain transient and/or stable expression of proteins of interest under the control of tissue-specific promoters.

Expansion and Further Propagating of Endodermal Progenitor Cells of Type B

The expansion of the endodermal progenitor cells of type B may be on feeder cells or in a feeder-free culture system.

When endodermal progenitor cells of type B are cultured in the presence of feeder cells, the culture medium from the initial differentiation may switched to an mesendodermal progenitor promoting medium supplemented with factors such as HGF, ActivinA or nodal and/or combinations thereof. Subsequent medium changes can be done between every 2 and 8 days, such as between every 3 and 7 days, such as between every 3 and 6 days, such as between every 4 and 5 days and the cells cultured under this conditions for between 10 and 28 days, such as between 12 and 26 days, such as between 14 and 24 days, such as between 16 and 22 days, such as between 18 and 20 days. Immuncytochemical analysis can be performed after fixation.

In one embodiment of the invention the hBS cells were cultured on mouse EF cells without passage or medium changes for 7 days. The medium was thereafter changed to an endoderm-promoting medium comprising either activin A, nodal or HGF. The promoting medium was then changed twice a week. Fractions from the resulting populations were fixed and analysed with immunohistochemistry at different time points.

The progenitor cells obtained after step B-1) and/or B-2) can optionally be dissected and re-plated on fresh feeder after from about 2 to about 14 days in culture, such as after from about 3 to 13 days, after from about 4 to about 12 days, after from about 5 to about 11 days, after from about 6 to about 10 days, after from about 7 to about 9 days, such as after about 8 days in culture. The dissection may be performed using any convenient instrument, such as a pipette or glass capillary. The subsequent passage (step B-3) may be performed using a chelator or enzymatic treatment after 2 to 14 days, such as after 3 to 13, 4 to 12, after 5 to 11, after 6 to 10, after 7 to 9, such as after 8 days in culture and the cells further transferred to fresh feeder, any culture supporting matrix or plastic. Subsequent passages can be performed enzymatic treatment after from about 2 to about 14 days, such as after from about 3 to about 13, after from about 4 to about 12 days, after from about 5 to about 11 days, after from about 6 to about 10 days, after from about 7 to about 9 days, such as after about 8 days in culture.

When endodermal progenitor cells of type B are cultured in a feeder-free culture system, the culture medium can be switched to a mesendodermal progenitor promoting medium supplemented with factors such as HGF, Activin A and/or nodal. Subsequent medium changes can be done between every 2 and 8 days, such as between every 3 and 7 days, such as between every 3 and 6 days, such as between every 4 and 5 days and the cells cultured under this conditions for between 10 and 28 days, such as between 12 and 26 days, such as between 14 and 24 days, such as between 16 and 22 days, such as between 18 and 20 days. Immunocytochemical analysis can be performed after fixation.

The progenitor cells obtained after step B-1) and/or B-2) can be transferred to a fresh culture system after 2 to 28 days in culture, such as after 4 to 24 days, after 6 to 20 days, after 8 to 16 days, after 9 to 14 days, after 11 to 12 days, such as after 8 days in culture. The transfer may be performed using any convenient instrument, such as a pipette or glass capillary or by enzymatic treatment or a chelator. Subsequent passages can be performed enzymatic treatment after 2 to 14 days, such as after 3 to 13, 4 to 12, after 5 to 11, after 6 to 10, after 7 to 9, such as after 8 days in culture.

In the expansion step B-2), one or more growth-promoting agents may be added. Those agents may be selected from the group consisting of activin A and HGF.

When activin A is used it is normally added to a concentration of from about 0.01 to 500 μg/ml such as, e.g., from about 0.05 to about 250 μg/ml, from about 0.1 to about 200 μg/ml, from about 1 to about 100 μg/ml or from about 10 to about 50 μg/ml such as about 25 μg/ml.

When HGF is used it is normally added to a concentration of from about 0.01 to 500 μg/ml such as, e.g., from about 0.05 to about 250 μg/ml, from about 0.1 to about 200 μg/ml, from about 1 to about 100 μg/ml or from about 10 to about 50 μg/ml such as about 20 μg/ml.

The fraction of the cells obtained in step B-2) that are endodermal progenitor cells of type B is at least 2.5%, such as, e.g., at least than 5%, or at least 10%, as evidenced in a sample of these cells.

The endodermal progenitor cells of type B may then be identified by positive reaction for a marker selected from the group consisting of Brachyury and HNF3beta such as by co-localization of the markers Brachyury and HNF3beta.

After the expansion and, if employed, the passaging of the mesendodermal progenitor cells, the population of mesendodermal progenitor cells is increased with a factor of at least 2 such as, e.g., a factor or 10 or more, a factor of 50 or more or a factor of 100 or more after step B-2) or B-3).

Differentiation of Endodermal Progenitor Cells of Type B to Hepatocyte-Like Cells

This step is essentially carried out as described in protocol A.

The fraction of the cells obtained in step B-4) that are hepatocyte-like cells is at least 5%, such as, e.g., at least 10%, at least 20%, at least 30%, at least 40%, at least 50%, at least 60%, at least 70%, at least 80%, at least 90% as evidenced in a sample of these cells.

The hepatocyte-like cells are identified by positive reaction for a marker selected from the group consisting of albumin, AFP, AAT, CK 18, LFABP, CYP and ASGPR. In particular, the hepatocyte-like cells are identified by positive reaction for at least one of the following markers, such as, e.g., at least two of the following markers, at least three of the following markers, at least four of the following markers, at least five of the following markers, at least six of the following markers: albumin, AFP, AAT, CK 18, LFABP, CYP and ASGPR. Especially, the hepatocyte-like cells are identified by positive reaction for albumin, AFP, AAT, CK 18 and LFABP.

Further characterization such as gene profiling and functional tests based on e.g. liver specific enzymatic activity can also be applied as well as transplantation of the endodermal progenitor cells and/or the hepatocyte-like cells to hepatectomic mice.

The major phase 1 biotransformation or metabolising system is the cytochrome P 450 (CYP). CYP expression in the different cell types obtained in the present invention can be analyzed on protein level by immunohistochemistry and/or Western Blot. An additional method is to genetically analyze the cells obtained in the present invention using real time PCR. Specific CYP subtypes of interest can be CYP 3A4 and CYP 1A2 (abundant in human liver), and CYP 3A7 (expressed in fetal liver tissue). CYP induction can then be tested by adding different known inducers, such as e.g. dexamethasone, rifampicin, and/or omeprazole.

The progenitor cells and hepatocyte-like cells obtained may further be analyzed for Glutathione transferases (GSTs). GSTs are phase 11 biotransformation enzymes that catalyze the conjugation of electrophilic xenobiotics with glutathione (GSH). Since GSTs have a wide range of substrates and GSH is highly abundant, GSTs are important players in detoxification of xenobiotics. Analysis of Phase II enzyme induction can be performed by any suitable method, such as immunofluorescence, confocal microscopy and/or possibly western blot analysis.

Multidrug resistance protein or P-glycoprotein is a transport protein that exports anionic conjugates and other substrates from the cell. Characterization of this third phase of the detoxification process could contribute to a more complete picture of metabolism of the different cell types obtained in the present invention.

The hepatocyte-like cells and/or intermediary progenitor cells, such as, e.g., endodermal progenitors of type A or B, obtained in the present invention may as well be characterized by analyzing their expression profile of metabolically significant genes. In the following is described how this can be done. RNA can be isolated from undifferentiated and differentiated hBS cells, such as endodermal progenitor cells and hepatocyte-like cells and, as a positive control, from human liver RNA. RNA extracted from a hepatic cell line, such as HepG2 may as well be used as a control. An expression profile can then be obtained comparing many different genes by using any suitable genetic tool for analysis, such as micro arrays, followed by bioinformatics analysis using a suitable software. The different cell types of the present invention may then show more or less expression of key genes, i.e. any genes which are specifically expressed in human adult liver, such as genes encoding for liver specific enzymes and transporter proteins. In addition the expression of the liver specific genes albumin and glucose-6-phosphatase can be analyzed for and possibly compared to the expression levels of the control samples.

The expression profiles and levels of specific genes or markers important for hepatocytes can be measured by e.g. RNA extraction and subsequent quantitative PCR, whereby the amount of any specific hepatic marker can be compared to control samples of e.g. traditionally used hepatic cell lines, such as, e.g., such as HepG2, and adult human hepatocytes. Hepatocyte-like cells obtained from endodermal progenitors of type B may show a profile in terms of which genes that are expressed and the expression levels of those genes similar to healthy, adult human hepatocytes. Specifically they may share the same profile of markers expressed, such as e.g. at least 20%, at least 30%, at least 40%, at least 50%, at least 60%, at least 70%, at least 80%, at least 90%, at least 95% of the markers expressed in healthy, adult (human) hepatocytes.

The expression levels of the individual markers may further constitute at least 2%, such as, e.g., at least 5%, at least 10%, at least 20%, at least 30%, at least 40%, at least 50%, at least 60%, at least 70%, at least 80%, at least 90%, at least 95% of the levels expressed in healthy adult hepatocytes cultured under the same conditions.

Hepatocyte-like cells obtained from endodermal progenitors of type B may display a more hepatocyte-like expression profile than hepatocyte-like cells obtained from endodermal progenitors of type A, in terms of the number of liver specific markers that are expressed and the expression levels of these markers. Accordingly, hepatocyte-like cells obtained from endodermal progenitors of type B may express more than 1/30 times more, such as, e.g., more than 1/20 times more, more than 1/10 times more, more than ⅕ times more, more than ¼ times more, more than ⅓ times more, more than ½ times more of the markers expressed by hepatocyte-like cells obtained from endodermal progenitors of type B (in absolute numbers). Furthermore, the expression levels of the individual markers expressed by hepatocyte-like cells obtained from progenitors of type B may be more than 1.2 times higher, such as, e.g., more than 1.5 times higher, more than 2 times higher, more than 5 times higher, more than 7.5 times higher, more than 10 times higher, more than 20 times higher, more than 50 times higher than the expression levels of the individual markers expressed by hepatocyte-like cells obtained from endodermal progenitor cells of type A.

The fraction of the cells obtained in B-4) that are undifferentiated BS cells is less than 2%, such as, e.g., less than 1%, less than 0.5% as evidenced in a sample of these cells.

The undifferentiated BS cells can be identified by positive reaction for a marker selected from the group consisting of Nanog, SSEA-3, SSEA-4, GCTM-2, Tra1-60, Tra1-81 and Oct-4 (this also applies for other steps in the method according to the method, where it is of relevance to identify any undifferentiated BS cells).

In a specific embodiment undifferentiated BS cells are identified by positive reaction for at least one of said markers, such as, e.g., at least two of said markers, at least three of said markers, at least four of said markers, at least five of said markers of the undifferentiated BS cells are identified by positive reaction for Nanog, SSEA-3, SSEA-4, GCTM-2, Tra1-60, Tra1-81 and Oct-4. In a specific embodiment the undifferentiated BS cells are identified by positive reaction for SSEA-3, SSEA-4, GCTM-2, Tra1-60, Tra1-81, Nanog and Oct-4.

The overall yield determined as the percentage of the number of hepatocyte-like cells obtained in proportion to the number of cells subjected to the method is at least 5%, such as, e.g., at least 10%, at least 20%, at least 30% or at least 40%.

When applying protocol B, the cells grow in two-dirnensional cultures.

In order to determine whether the cells obtained from any of the steps in B-1) to B-3) are committed to an endodermal cell fate, the cells have to fulfil several criterion based on imuncytochemistry and morphology. To determine whether the cells express different cell type specific markers like the endodermal progenitor, HNF3beta, gata4, Cdx2, Sox 17 and Pdx1 or liver cell marker, Albumin, alpha-fetoprotein, alpha-1-antitrypsin, cytokeratin18, LFABP, ASPGR. Another test is to determine whether the cells still express markers for undifferentiated hBS cells, such as, e.g. the markers Nanog, SSEA-3 (stage specific embryonic antigen 3), SSEA-4, GCTM-2, Tra-1-60, Tra-1-81 (tumour rejection antigens), Oct-4.

Spontaneous differentiation generates the variety of cells that can respond to the growth factors or low molecular compounds that we have used. Definitive-resembling endoderm arises later from a mesendodermal cell population that can be defined by HNF3beta-Brachyury double positive cells. That population can respond to ActivinA or HGF (Kubo et al., 2004) for induction and maintenance of the cell fraction.

In one embodiment of the invention, the cells obtained from step B-1 and/or B-2) and/or B-3) have at least one of the following properties:
I) a majority of the cell population expressing co-localization of HNF3b/Brachyury markers
II) a majority of the cell population expressing at least one of the following markers HNF3beta, Gata4, Pdx1, Sox 17
III) a majority of the cell population being capable of further differentiation with following
   a) expression of at least one of the following markers Albumin, HNF3beta, LFABP, Ck18, AFP, alpha-1-antitrypsin, ASGPR
   b) liver enzyme activities such as for Cytochrome P450
IV) a majority of the cell population being unable to express one or more of the following markers for undifferentiated hBS cells, Nanog, SSEA-3, SSEA-4, GCTM-2, Tra1-60, Oct-4 and Tra1-81.

In the present context, the term “majority of cells” is intended to denote at least about 60% of the cells such as, e.g., at least about 75%, at least about 90% or at least about 95% of the cells.

In one embodiment of the invention, the majority of the cells obtained from step B-1) and/or B-2) and/or B-3i), do not express markers for the ectoderm, such as, e.g., GFAP or/and nestin.

The cells obtained from step B-2)-B-4), might be further differentiated into at least one of the liver cell lineages, e.g., oval cells or hepatocytes.

In one embodiment of the invention, the differentiated cells obtained in B-4) may express at least one of the following liver cell type markers, including at least one of the markers HNF3beta, albumin, AFP, AAT, CK18, LFABP, ASGPR.

Growth Media

In the above discussion of the initial differentiation of BS cells, the employment of the protocols A or B the use of culture media or growth media is described.

The base medium used for the generation of embryonic progenitors and further hepatocyte-like cells from hBS cells may be any suitable growth medium, such as, e.g. hBS cell medium, VitroHES™-medium or Hepatocyte medium and DMEM/F12 based medium. The growth medium used in the different steps of a method of the invention may be the same or different and depends on factors included. All of these media may be used as conditioned media, such as, e.g. k-hBS medium, k-VitroHES™-medium. The preferred base medium throughout the invention is VitroHES™-medium (Vitrolife, Gothenburg, Sweden) or alternatively a medium termed “hBS-medium” which may be comprised of; KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0.1 mM non-essential amino acids, 2 mM L-glutamine, 100 μM β-mercaptoethanol (all ingredients from Invitrogen) or Hepatocyte medium (Invitrogen).

Growth Additives

Growth media for use in the method of the present invention may comprise one or more growth factors or combinations of them. The growth factors used may be any suitable growth factors for the generation of endodermal progenitor cells of type B. The concentration of the specific growth factor used may be important for whether the cells will differentiate further or remain in the progenitor state. Specific examples of a growth factor usable for promoting the generation and propagation of endodermal progenitors of type B are HGF, Activin A, and Nodal, whilst FGF2 can be used for the initial in vitro differentiation (step (i)). A cytotoxic compound such as DMSO can be used in the later stages for further differentiation towards hepatocyte-like cells.

The amount of all growth additives to be used to promote generation and propagation of endodermal progenitor& cells of type B may be from about 1 ng/ml to about 200 ng/ml, such as from about 0.5 ng/ml to about 100 ng/ml, from about 1 ng/ml to about 50 ng/ml, from about 2 ng/ml to about 30 ng/ml, from about 3 ng/ml to about 20 ng/ml, from about 4 ng/ml to about 12 ng/ml or from about 4 to about 8 ng/ml.

Other suitable growth additives or factors that promote the generation, propagation and expansion of the endodermal progenitor cells of type B can of course be used.

### Other Aspects of the Invention

In other aspects the present invention relates to the use of hepatocyte-like cells, obtained by the method described herein, such as e.g. use in medicine and more specifically for the prevention and/or treatment of pathologies or diseases caused by tissue degeneration, such as, e.g., the degeneration of liver tissue or for the prevention or treatment of metabolic pathologies and/or diseases. Examples of diseases and disorders, which may be prevented and/or treated by a medicament comprising hepatocyte-like cells, may be selected from different groups of liver disorders: 1) auto immune disorders such as primary biliary cirrhosis, 2) metabolic disorders, such as dyslipidemia, 3) liver disorders caused by e.g. alcohol abuse, 4) diseases caused by viruses such as hepatitis B, -C, and, -A, 5) liver necrosis caused by acute toxic reactions to e.g. pharmaceutical drugs, and 6) tumor removal in patients suffering from e.g. hepatocellular carcinoma.

In still other aspects, the invention relates to the use of obtained hepatocyte-like cells in in vitro models for studying hepatogenesis, such as, e.g., early hepatogenesis or in in vitro models for human hepatoregenerative disorders.

Furthermore, the invention relates to use of obtained hepatocyte-like cells in a drug discovery process and for hepatotoxicity testing in vitro in order to replace or complement to conventional model systems.

In further aspects the present invention relates to methods for treatment of hepatocyte-susceptible disorders or conditions of an animal including a human by administration an effective amount hepatocyte-like cells obtained according to the invention. Such a hepatocyte-susceptible disorder or condition may be a liver disorder, such as, e.g., auto immune disorders including primary biliary cirrhosis; metabolic disorders including dyslipidemia; liver disorders caused by e.g. alcohol abuse; diseases caused by viruses such as, e.g., hepatitis B, -C, and, -A; liver necrosis caused by acute toxic reactions to e.g. pharmaceutical drugs; and tumor removal in patients suffering from e.g. hepatocellular carcinoma.

The invention also relates to a composition of endodermal progenitor cells obtained in step ii). In such a composition the cells obtained may exhibit at least one of the endodermal progenitor cell type markers selected from the group consisting of HNF3beta, Pdx1, gata4, Cdx2 and Sox 17 and without the majority of the cells expressing one or more markers for undifferentiated hBS cells, from the group consisting of Nanog, SSEA-3, SSEA-4, GCTM-2, Tra-1-60 or Tra-1-80.

Another embodiment relates to a preparation of hepatocyte-like cells obtained by a method as described herein, wherein the amount of hepatocyte-like cells may be at least 50% of the total cell population, such as, e.g. at least 60%, at least 70%, at least 80%, at least 90%, at least 95%, at least 96%, at least 97%, at least 98%, at least 99% or 100%.

The differentiated cells may display the morphology and expression criteria of at least one for the following liver markers AAT, AFP, LFABP, Ck18, Albumin, HNF3beta and ASGPR.

In another aspect, the progenitor cells are differentiated into hepatocyte-like cells, which may be characterized by the presence of the cell markers AAT, AFP, LFABP, CK18, Albumin, HNF3beta and ASGPR.

In still another aspect the invention relates to methods to separate the endodermal and hepatocyte-like populations from other cell types obtained, such as e.g. separating the extraembryonic endodermal cells obtained in step (i) from the other cell types possibly present by using a suitable combination of antigen markers.

The invention also relates to methods for detecting the effect of the progenitor cells or the hepatocyte-like cells obtained from those on the concentration of a chemical substance added and its metabolites at different time points.

In still further aspects the present invention relates to use of the hepatocyte-like cells in drug discovery and drug development for screening of molecular substances as a target to monitor hepatic differentiation. Also the intermediary endodermal progenitor cells of type A and/or B obtained herein may be used as a target to study hepatic maturation by the exposure of certain chemicals.

The hepatocyte-like cells may in still further aspects be used in metabolic studies by analysing the phase 1 biotransformation or metabolising system, i.e. the cytochrome P 450, the phase II biotransformation enzymes that catalyze the conjugation of electrophilic xenobiotics with glutathione (GSH),and/or multidrug resistance protein or P-glycoprotein which is a transport protein that may export anionic conjugates and other substrates from the cell as described herein.

The hepatocyte-like cells obtained may as well be used for toxicity typing of certain chemical compounds of interest.

The different use aspects of the present invention listed herein may of course be performed in any suitable format such as low, medium and possibly high through-put. Preferred is a multi-well format compatible with automation.

Further aspects and embodiments appear from the appended claims. The details and particulars discussed under the main aspects above apply mutatis mutandis to the other aspects of the invention.

### THE INVENTION IS FURTHER ILLUSTRATED BY THE FOLLOWING FIGURES

FIG. 1: A scheme describing the early endodermal development.

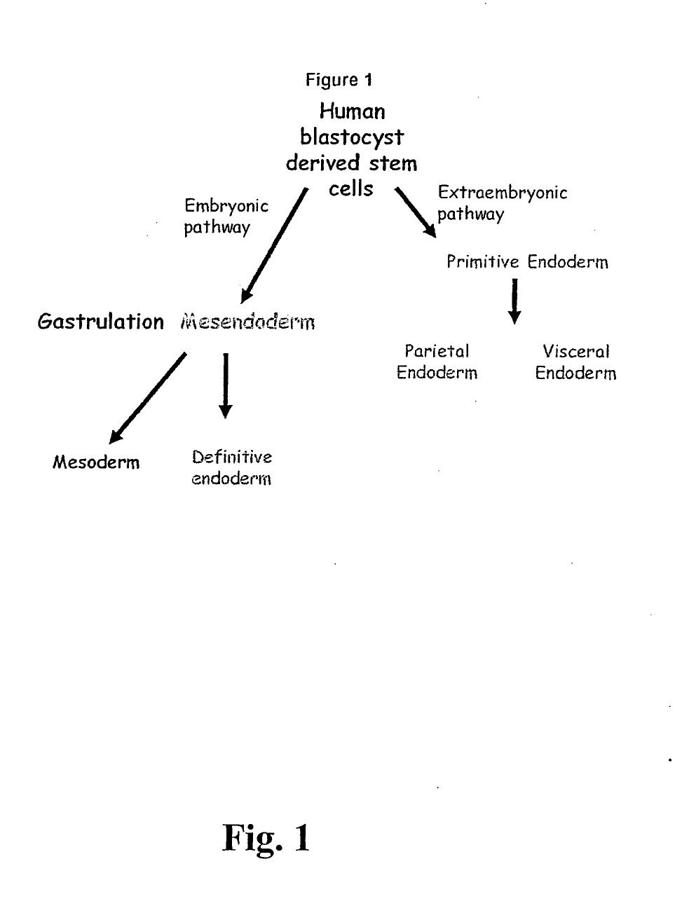

FIG. 2: A flow chart describing the method for generation of hepatocyte-like cells from undifferentiated hBS cells via endodermal progenitor cells of type A and B.

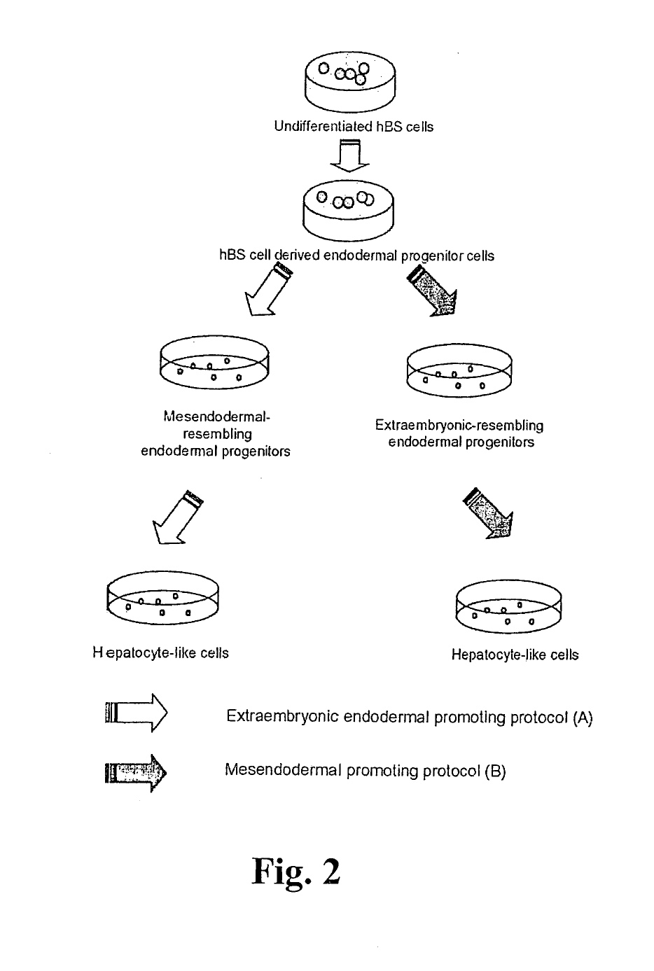

FIG. 3: Hepatocyte-like cells from hBS cell line SA002 at day 16 using protocol (A). Morphology is shown in A, positive reactions for HNF3beta in B, LFABP in C, Albumin in D, AAT in E, CK18 in F.

FIG. 4: Endodermal progenitor cells of type B after 14 days in culture. A) Brachyury and B) HNF3b positive (protocol (B)).

FIG. 5: Endodermal progenitor cells of type A after 12 days in culture. A) Oct-4 and B) Pdx-1 positive (protocol A).

The invention will now be described with reference to the following examples. The examples are included herein for illustrative purposes only and are not intended to limit the scope of the invention in any way. The general methods described herein are well known to a person skilled in the art and all reagents and buffers are readily available, either commercially or easily prepared according to well-established protocols in the hands of a person skilled in the art. All incubations were in 37° C, under a 5% CO₂ atmosphere and 95% humidity.

### EXAMPLES

In PCT application published as WO 03/055992 (to the same Applicant) on 10 Jul. 2003 a suitable method for establishing hBS cells is described. In one aspect of the present invention, the cells employed in the examples herein are obtained by the method claimed in WO 03/055992, which is hereby incorporated by reference.

### Example 1

Generation of Endodermal Progenitor Cells of Type A from hBS Cells Cultured on Mouse EF Cells

hBS cells cultured on mouse EF cells in VitroHES™ (Vitrolife AB, Kungsbacka, Sweden) supplemented with 4 ng/ml bFGF (FGF2) (Invitrogen) were left to differentiate without medium changes for 5 to 7 days. The medium was thereafter switched to an extraembryonic promoting medium, e.g. VitroHES™ without FGF2 supplemented with 4 ng/ml of RA and the cells cultured under this conditions for seven days. The medium was changed once during culture.

Another extraembryonic promoting medium used was VitroHES™ supplemented with 20 ng/ml BMP2. The cells were cultured for seven days in this medium and the medium changed once.

### Example 2

Generation of Endodermal Progenitor Cells of Type A from hBS Cells Cultured on Matrigel™

hBS cells cultured on Matrigel™ (BD Biosciences) in VitroHES™ medium supplemented with 4 ng/ml bFGF (FGF2) were left to differentiate without medium changes for 12 to 14 days. The medium was thereafter switched to an extraembryonic promoting medium, e.g. VitroHES™ without FGF2 supplemented with 4 ng/ml of RA and the cells cultured under this conditions for 14 days. The medium was changed 6 times during this culture.

### Example 3

Generation of Epithelial Endodermal Cell Line from Extraembryonic Endoderm

Progenitor cells obtained in Example 1 above were mechanically dissected and re-plated on fresh feeder after 7 days. The cells were further passaged after 7 days using Trypsin-EDTA, 0.05M (Gibco) for 3 minutes and the cell suspension was washed and centrifuged once (170 g, 5 minutes) and transferred to cell culture flasks in VitroHES™ (+bFGF, 4 ng/ml). The cell was thereafter passaged every 3 to 4th day for more than 10 passages.

Freezing and Thawing of the Cell Line Obtained

The cell suspension was collected, diluted in culture medium (37° C.), pelleted, washed in culturing medium (37° C.) and resuspended in freeze-medium (4 to 8° C.). The freeze-medium consisted of culturing medium supplemented with 10% DMSO. The cells were frozen at a cell density of one million cells/mL. The cell suspension was aliquoted in 1.8 mL Nunc CryoTubes (Nalge Nunc International, Rochester, N.Y.) and frozen slowly (−1° C./min) at −80° C. overnight or at least for 2 h, then transferred to a liquid nitrogen tank for prolonged storage. Thawing of the cells was done by a rapid thawing by placing the CryoTubes in 37° C. water bath until completely thawed, transferring the suspension to preheated (37° C.) culture medium for 5 min, spin down the cells (400 g, 5 min), wash in culture medium (37° C.) and resuspension in culture medium. The thawed cells were then seeded, as described above for propagation of progenitor cells.

### Example 4

Generation of Hepatocyte-Like Cells by Differentiation of Progenitor Cells Obtained in Example 1

To the cell population obtained in Example 1 a differentiation medium, VitroHES™ containing 1% DMSO, was added in which the cells were cultured for 7 days without medium changes.

### Example 5

Generation of Hepatocyte-Like Cells by Differentiation of Progenitor Cells Obtained in Example 2

To the cell population obtained in Example 2 a differentiation medium containing 1% DMSO, in which the cells were cultured for 14 days with one medium change.

### Example 6

Generation of Hepatocyte-Like Cells by Differentiation of hBS Cells Via Endodermal Progenitor Cells of Type B

hBS cells co-cultured with mouse embryonic fibroblasts were left to differentiate in VitroHES™ with 4 ng/mL FGF-2 for seven days without medium change. To induce definitive endoderm VitroHES™ without FGF2 was supplemented with ActivinA (R&D Systems) 5 ng/ml or HGF (Chemicon International) 8 ng/ml. Medium was changed twice a week. After five to seven days the medium was changed to a VitroHES™ based medium without growth factors and 1% DMSO and kept for additional five days with one medium change. Half the medium volume was changed twice a week. After five to seven days the medium was changed to VitroHES™ supplemented with 1% DMSO and kept for additional five days with one half-volume medium change.

### Example 7

Immunocytochemical Characterization of Cells Obtained

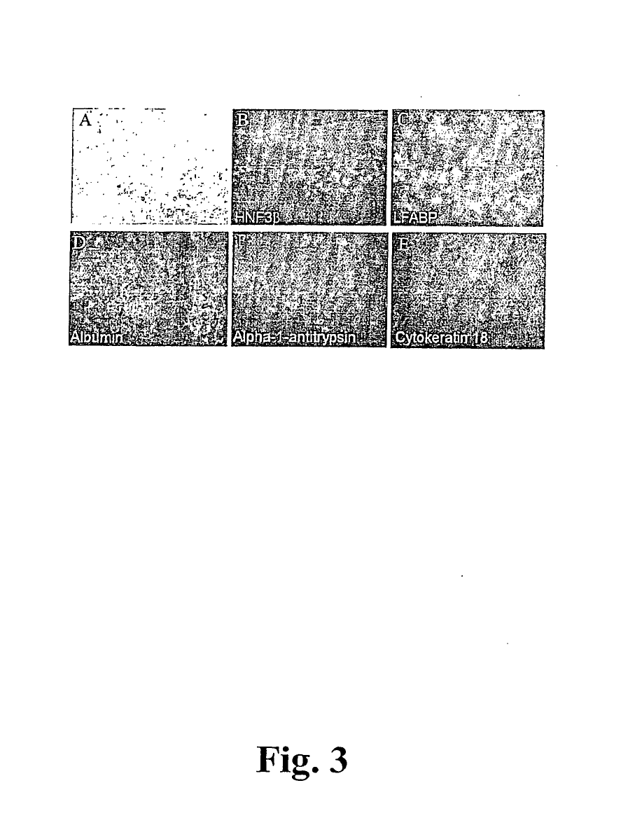

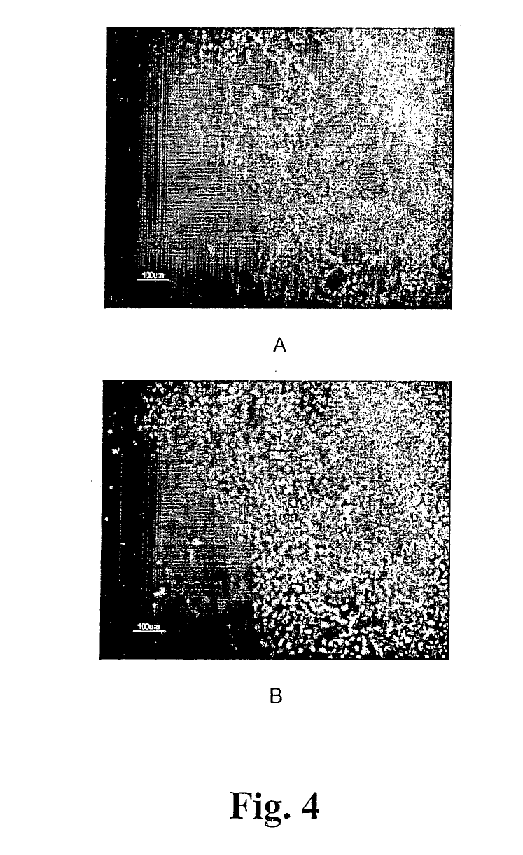

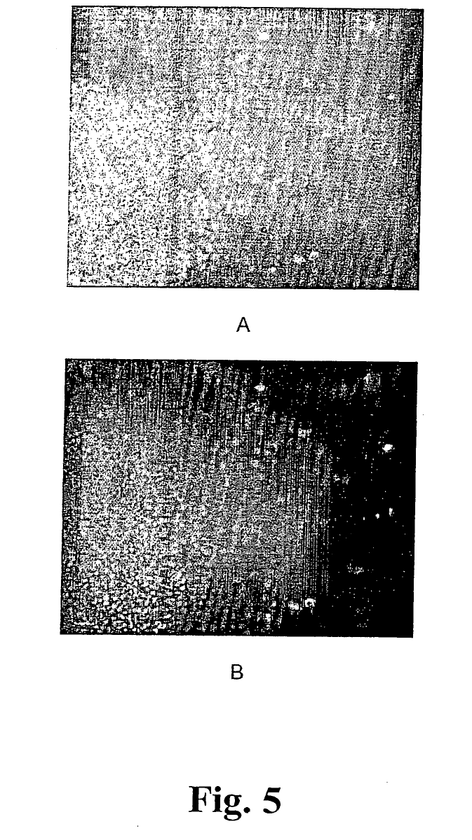

(See FIGS. 3 to 5.)

The following markers were used, for:

| | |
|---|---|
| Endodermal progenitor cells of type A: | combination of Oct4 and Pdx1 |
| Endodermal progenitor cells of type B: | combination of Brachyury and |
| | HNF3b |
| hepatocyte-like cells derived via | AAT, CK 18, AFP, LFABP and |
| endodermal progenitors cells of type | albumin |
| A or B: | |
| undifferentiated hBS cells: | Oct4, SSEA-4, Tra-1-60 and |
| | Tra-181 |

Washes and dilutions were done in D-PBS (Gibco), fixation in 4% PFA (Histolab), permeabilization in 0.5%Triton X-100 (Sigma). Fluorescent Mounting Medium (DAKO) was used for mounting samples and nuclear stainings were performed with 0.5 □g/ml DAPI (Sigma).

Primary antibodies were incubated over night at 4° C. and the secondary antibody together with DAPI for between 40 and 60 minutes dark at room temperature.

Dilutions and Conjugating Secondary Antibodies:

SSEA-4 (1:200) (Developmental Studies Hybridoma Bank)—mouse IgG (Southern Biotech)(1 :200)−FITC

Tra1-60 (1:250) (Developmental Studies Hybridoma Bank)—mouse IgM (Southern Biotech), (1:200)−Rhodamine

Tra1-81 (1:250) (Developmental Studies Hybridoma Bank)—mouse IgM (Southern Biotech), (1:200)−Rhodamine

Oct 4, (1:500) (Developmental Studies Hybridoma Bank)—mouse IgG 2b (Southern Biotechnology Associates), (1:50)−FITC

Brachyury, (1:1000), rabbit IgG (access through academic collaboration), (1:500)—Rhodamine

or:

Brachyury, (1:1000)—rabbit IgG ( ), (1:500) (Santa Cruz Biotechnology)—rabbit-Cy3 (Jackson ImmunoResearch Laboratories)

HNF3beta, (1:500)—goat IgG, (1:500)—Streptavidin (DAKO/Vector Laboratories) (1:250)—FITC

Pdx1, (1:1000) (access through academic collaboration); rabbit IgG (Jackson ImmunoResearch Laboratories and/or DAKO), 1:500—Rhodamine

or:

Pdx1, (1:500) (ABCAM, (ab 19379); rabbit IgG (Jackson ImmunoResearch Laboratories or DAKO), 1:500—Rhodamine

AAT (1:200) (ABCAM)—rabbit IgG (Jackson lmmunoResearch Laboratories or DAKO), (1:500)—Rhodamine

CK18 (1:200)(DAKO)—mouse IgG (Southern Biotech)(1:200)—FITC

Albumin (1:50) (DAKO)—rabbit IgG (Jackson ImmunoResearch Laboratories or DAKO), (1:500)—FITC

LFABP (1:250) (Santa Cruz)−goat IgG, (1:500), +Streptavidin (DAKO/Vector Laboratories), (1:250)—Rhodamine/FITC

AFP (1:1500) (Sigma)−mouse IgG2A (Southern Biotech), (1:100)—Rhodamine

Result:

After approximately 14 days culture on feeder layers according to the protocol described previously all hepatocyte-like markers were expressed for hepatocyte-like cells obtained in both protocols.

### REFERENCES

Wells, J. M. and Melton, D. A. Early mouse endoderm is patterned by soluble factors from adjacent germ layers. Development. 2000, 127(8):1563-72.

Coucouvanis E. and Martin G. R. BMP signaling plays a role in visceral endoderm differentiation and cavitation in the early mouse embryo. Development. 1999;126(3):535-546.

[Brolén, GKC, Heins. N, Edsbagge. J, and Semb. H, Signals From the Embryonic Mouse Pancreas Induce Differentiation of Human Embryonic Stem Cells Into Beta-Cell-Like Insulin-Producing Cells, Diabetes 54, 2005]

Goldin, S. N. and Papaioannou, V. E. (2003). Paracrine action of FGF4 during periimplantation development maintains trophectoderm and primitive endoderm. Genesis; 36(1):40-47.

Kubo A., Shinozaki, K., Shannon, J. M., Kouskoff, V., Kennedy, M., Woo, S., Fehling, H. J. and Keller, G. (2004). Development of definitive endoderm from embryonic stem cells in culture. Development; 131(7): 1651-1662.

Rambhatla, L., Chiu, C. P., Kundu, P., Peng, Y. and Carpenter, M. K. (2003). Generation of hepatocyte-like cells from human embryonic stem cells. Cell Transplant.; 12(1):1-11.

Smedberg, J. L., Smith, E. R., Capo-Chichi, C. D., Frolov, A., Yang, D. H., Godwin, A. K., Xu, X. X. (2002). Ras/MAPK pathway confers basement membrane dependence upon endoderm differentiation of embryonic carcinoma cells. J Biol Chem.; 25;277(43):40911-40918.

US2003003573, Hepatocytes for therapy and drug screening made from embryonic stem cells, Carpenter, M. K et al

U.S. Pat. No. 6,506,574, Hepatocyte lineage cells derived from pluripotent stem cells, Carpenter, M. K. et al

U.S. Pat. No. 6,458,589, Hepatocyte lineage cells derived from pluripotent stem cells, Carpenter, M. K et al

1. A method for obtaining endodermal progenitor cells and further differentiating these to hepatocyte-like cells comprising the steps:
i) In vitro differentiating BS cells in a growth medium comprising FGF 2 to obtain differentiated cells of which at least a fraction of them are endodermal progenitor cells,
ii) determining the fraction of the endodermal progenitor cells obtained in step i) being endodermal progenitors of type A and/or endodermal progenitors of type B,
iii) optionally, determining the fraction of the cells obtained in step i) being undifferentiated BS cells,
iv) optionally, selecting either endodermal progenitor cells of type A or endodermal progenitor cells of type B from the cells obtained in step i),
v) subjecting the endodermal progenitor cells of known composition obtained in steps i) or iv) to protocol A comprising the following steps:
A-1) subjecting the endodermal progenitor cells of known composition obtained in steps i) or iv), if relevant, to a growth medium and optionally, changing the growth medium after suitable period(s) of time,
A-2) expanding the endodermal progenitor cells of known composition obtained in steps i), iv) or A-1) by addition of one or more growth-promoting agents selected from the group consisting of RA, FGF4, and BMP2,
A-3) optionally, passaging the cells obtained in steps i), iv) or A-2) one or more times leading to further expansion of said cells,
A-4) inducing differentiation of the progenitor cells obtained in steps i), iv), A-2) or A-3) by addition of one or more differentiating agents that are liver enzyme inducing agents selected from the group consisting of DMSO, ethanol, dexamethasone, Phenobarbital and urea, to obtain hepatocyte-like cells.

2. A method according to claim 1, wherein the BS cells are hBS cells.

3. A method according to claim 1, wherein step iv) is included.

4. A method according to claim 1, wherein the fraction of endodermal progenitor cells of type A obtained in steps i) or iv) is larger than the fraction of endodermal progenitor cells of type B obtained in steps i) or iv).

5. A method according to claim 1, wherein the cells in steps i)-v) or A-1)-A-4) are cultured in a 2 dimensional culture comprising a surface to which the cells adhere.

6. A method according to claim 1, wherein the fraction of the cells obtained in step i) and/or step iv) that are endodermal progenitor cells of type A is at least 10% as evidenced in a sample of these cells.

7. A method according to claim 1, wherein the fraction of the cells obtained in step i) and/or step iv) that are undifferentiated BS cells is less than 85% as evidenced in a sample of these cells.

8. A method according to claim 1, wherein the endodermal progenitor cells of type A obtained in step i) are selected by inclusion of step iv).

9. A method according to claim 8, wherein the endodermal progenitor cells of type A are selected by
a) using neomycin selection in culture and/or
b) using flow cytometry.

10. A method according to claim 1, wherein FGF 2 is added to a concentration from about 0.1 ng/ml to about 200 ng/ml.

11. A method according to claim 1, wherein the one or more growth-promoting agents in step A-2) are added to a concentration of from about 0.1 ng/ml to about 1000 ng/ml.

12. A method according to claim 1, wherein the fraction of the cells obtained in step A-2) that are endodermal progenitor cells of type A is at least 20% in a sample of these cells.

13. A method according to claim 1, wherein the endodermal progenitor cells of type A are identified by positive reaction for Oct-4.

14. A method according to claim 1, wherein the endodermal progenitor cells of type A are identified by positive reaction for a marker selected of the group consisting of HNF3beta, Gata4, Cdx2, Sox17 and Pdx1.

15. A method according to claim 1, wherein the endodermal progenitor cells of type A are identified by positive reaction for HNF3beta, Gata4, Cdx2, and Pdx1.

16. A method according to claim 1, wherein the endodermal progenitor cells of type A are identified by a positive reaction for Oct-4 in combination with any of the following markers: HNF3beta, Gata4, Cdx2, Sox17 and Pdx1.

17. A method according to claim 1, wherein step A-3) is included.

18. A method according to claim 1, wherein the population of endodermal progenitor cells of type A is increased with a factor of at least 2 after step A-2) or A-3).

19. A method according to claim 1, wherein the one or more differentiating agents of step A-4) are one or more toxic agents.

20. A method according to claim 19, wherein said toxic agent is degradable by the liver.

21. A method according to claim 1, wherein DMSO in step A-4) is added to a concentration from about 0.5% to about 10%.

22. A method according to claim 1, wherein the differentiating agent in step A-4) is an alcohol.

23. A method according to claim 1, wherein the fraction of the cells obtained in step A-4) that are hepatocyte-like cells is at least 5% in a sample of these cells.

24. A method according to claim 23, wherein the hepatocyte-like cells are identified by positive reaction for a marker selected of the group consisting of albumin, AFP, MT, CK 18, LFABP, CYP and ASGPR.

25. A method according to claim 24, wherein the hepatocyte-like cells are identified by positive reaction for at least one of the following markers: albumin, AFP, AAT, CK 18 LFABP, CYP and ASGPR.

26. A method according to claim 24, wherein the hepatocyte-like cells are identified by positive reaction for albumin, AFP, MT, CK 18 and LFABP.

27. A method according to claim 1, wherein the fraction of the cells obtained in step A-4) that are undifferentiated BS cells is less than 2% in a sample of these cells.

28. A method according to claim 1, wherein the undifferentiated BS cells are identified by positive reaction for a marker selected from the group consisting of SSEA-3, SSEA-4, GCTM-2, Tra1-60, Tra1-81, Nanog, and Oct-4.

29. A method according to claim 28, wherein the undifferentiated BS cells are identified by positive reaction for at least two of said markers.

30. A method according to claim 28, wherein the undifferentiated BS cells are identified by positive reaction for SSEA-3, SSEA-4, GCTM-2, Tra1-60, Tra1-81, Nanog, and Oct-4.

31. A method according to claim 1, wherein the overall yield determined as the percentage of the number of hepatocyte-like cells obtained in proportion to the number of cells subjected to the method is at least 20%.

32. A method for obtaining hepatocyte-like cells comprising the steps:
A-i) in vitro differentiating BS cells in a growth medium comprising FGF2 to obtain differentiated cells of which at least a fraction of them endodermal progenitor cells of type A,
A-ii) optionally, selecting the endodermal progenitor cells of type A
A-iii) subjecting the endodermal progenitor cells of type A obtained in steps i) or ii) to protocol A comprising the following steps:
A-1) subjecting the endodermal progenitor cells of known composition obtained in steps i) or iv), if relevant, to a growth medium and optionally, changing the growth medium after suitable period(s) of time,
A-2) expanding the endodermal progenitor cells of known composition obtained in steps i), iv) or A-1) by addition of one or more growth-promoting agents selected from the group consisting of RA, FGF4, and BMP2,
A-3) optionally, passaging the cells obtained in steps i), iv) or A-2) one or more times leading to further expansion of said cells,
A-4) inducing differentiation of the progenitor cells obtained in steps i), iv), A-2) or A-3) by addition of one or more differentiating agents that are liver enzyme inducing agents selected from the group consisting of DMSO, ethanol, dexamethasone, Phenobarbital and urea, to obtain hepatocyte-like cells.

33. A method according to claim 32, wherein the BS cells are hBS cells.

34. A method according to claim 32, wherein step A-ii) is included.

35-36. (canceled)

37. A method of modeling heptaogenesis comprising using hepatocyte-like cells obtained by a method according to claim 1 in in vitro models for studying hepatogenesis.

38. A method of modeling human hepatoregenerative disorders comprising using of hepatocyte-like cells obtained by a method according to claim 1 in in vitro models for studying human hepatoregenerative disorders.

39. A method of screening molecular substances in drug discovery, comprising using hepatocyte-like cells obtained by a method according to claim 1 for screening of molecular substances as a target to monitor hepatic differentiation.

40. A method of hepatotoxicity testing comprising using hepatocyte-like cells obtained by a method according to claim 1 for in vitro hepatotoxicity testing.

41. A method of treatment and/or prevention of pathologies and/or diseases caused by tissue degeneration, comprising administering a effective amount of hepatocyte-like cells obtained by a method according to claim 1.

42-44. (canceled)

45. A method for treatment of a hepatocyte-susceptible disorder or condition of an animal including a human by the administration of an effective amount hepatocyte-like cells obtained by a method according to claim 1 to the animal in need thereof.

46. A method according to claim 45, wherein the hepatocyte-susceptible disorder or condition is a liver disorder.

47. A method according to claim 46, wherein the liver disorder is selected from the group consisting of auto immune disorders; metabolic disorders; liver disorders caused by alcohol abuse; diseases caused by viruses; liver necrosis caused by acute toxic reactions; and tumor removal.

48. A method according to claim 46, wherein the hepatocyte-susceptible disorder is a metabolic pathology and/or disease.
